(19) United States
(12) Patent Application Publication
(10) Pub. No.: US 2025/0005770 A1
(43) Pub. Date: Jan. 2, 2025

(54) MOVEMENT INFORMATION GENERATION APPARATUS, MOVEMENT INFORMATION GENERATION SYSTEM, MOVEMENT INFORMATION GENERATION METHOD, AND STORAGE MEDIUM

(71) Applicant: NEC Corporation, Minato-ku, Tokyo (JP)

(72) Inventors: Naoki SHINDOU, Tokyo (JP); Jianquan LIU, Tokyo (JP); Yuta NAMIKI, Tokyo (JP); Youhei SASAKI, Tokyo (JP)

(73) Assignee: NEC Corporation, Minato-ku, Tokyo (JP)

(21) Appl. No.: 18/711,254

(22) PCT Filed: Mar. 31, 2022
(86) PCT No.: PCT/JP2022/016540

§ 371 Date: May 17, 2024

(51) Int. Cl. G06T 7/246; G06T 7/00

(52) U.S. Cl. CPC G06T 7/248; G06T 7/0016; G06T 2207/20081; G06T 2207/30196

(57) ABSTRACT

A movement information generation apparatus (10) is an apparatus that assists rehabilitation carried out by a subject, and includes an image processing unit (120) and an output unit (130). The image processing unit (120) generates first movement information indicating movement of the subject at first timing by processing a first image acquired by capturing the subject at the first timing. The output unit (130) determines a difference between the first movement information and comparison movement information indicating movement being a comparison target, and outputs output information being information relating to the difference.

### TECHNICAL FIELD

The present invention relates to a movement information generation apparatus, a movement information generation system, a movement information generation method, and a storage medium.

### BACKGROUND ART

An opportunity to cause an injured or diseased individual to carry out rehabilitation has been increasing with an aim of restoring basic movement.

One of techniques for supporting rehabilitation is a rehabilitation movement evaluation apparatus described in Patent Document 1. The rehabilitation movement evaluation apparatus executes the following processing. First, the apparatus acquires two-dimensional moving image data acquired by capturing a user undergoing rehabilitation training. Subsequently, the apparatus estimates skeleton information indicating a skeleton of the user in the two-dimensional moving image data, based on the two-dimensional moving image data. Further, the apparatus evaluates an extent of rehabilitation training movement relating to rehabilitation training carried out by the user, based on the skeleton information being estimated. In Patent Document 1, one example of the evaluation includes whether the movement is correctly performed or incorrectly performed.

Further, Patent Document 2 describes the following learning apparatus. The learning apparatus is used together with a rehabilitation assisting apparatus. The rehabilitation assisting apparatus includes an actuator that assists rehabilitation movement of a trainee and a sensor that detects data relating to the rehabilitation movement assisted by the actuator, and transmits rehabilitation data to the learning apparatus. The rehabilitation data includes detection data according to a detection result of the sensor. The learning apparatus generates a learning model by performing machine learning that uses the rehabilitation data as learning data. The learning model uses the detection data as an input, and outputs an indicator indicating movement ability of the trainee.

### RELATED DOCUMENT

### Patent Document

Patent Document 1: Japanese Patent Application Publication No. 2021-049319
Patent Document 1: Japanese Patent Application Publication No. 2021-003489

### DISCLOSURE OF THE INVENTION

### Technical Problem

According to Patent Document 1 and Patent Document 2 described above, movement of a rehabilitation subject can be evaluated. However, in order to assist carrying out rehabilitation appropriately, only evaluation on movement of a subject is insufficient.

The present invention has been made in view of the above-mentioned circumstances, and one object thereof is to provide a movement information generation apparatus, a movement information generation system, a movement information generation method, and a storage medium that can assist carrying out rehabilitation of a subject appropriately.

### Solution to Problem

According to one aspect of the present invention, there is provided a movement information generation apparatus, including:
an image processing unit that generates first movement information indicating movement of a subject at first timing by processing a first image acquired by capturing the subject at the first timing; and
an output unit that determines a difference between the first movement information and comparison movement information indicating movement being a comparison target, and outputs output information being information relating to the difference.

According to one aspect of the present invention, there is provided a movement information generation system, including:
a terminal; and
a movement information generation apparatus, wherein
the movement information generation apparatus includes
   an image processing unit that generates first movement information indicating movement of a subject at first timing by processing a first image acquired by capturing the subject at the first timing, and
   an output unit that determines a difference between the first movement information and comparison movement information indicating movement being a comparison target, and outputs, to the terminal, output information being information relating to the difference, and
the terminal displays or prints the output information.

According to one aspect of the present invention, there is provided a movement information generation method including,
by a computer:
   generating first movement information indicating movement of a subject at first timing by processing a first image acquired by capturing the subject at the first timing; and
   determining a difference between the first movement information and comparison movement information indicating movement being a comparison target, and outputting output information being information relating to the difference.

According to one aspect of the present invention, there is provided a computer-readable storage medium storing a program causing a computer to execute:
image processing of generating first movement information indicating movement of a subject at first timing by processing a first image acquired by capturing the subject at the first timing; and
output processing of determining a difference between the first movement information and comparison movement information indicating movement being a comparison target, and outputting output information being information relating to the difference.

### Advantageous Effects of Invention

According to one aspect of the present invention, there can be provided a movement information generation apparatus, a movement information generation system, a movement information generation method, and a storage medium that can assist carrying out rehabilitation of a subject appropriately.

### BRIEF DESCRIPTION OF THE DRAWINGS

FIG. 1 is a diagram illustrating an overview of a movement information generation apparatus according to an example embodiment.

FIG. 2 is a diagram illustrating one example of a functional configuration of the movement information generation apparatus along with a usage environment of the movement information generation apparatus.

FIG. 3 is a diagram illustrating one example of skeleton information.

FIG. 4 is a diagram illustrating one example of information stored in a subject information storage unit.

FIG. 5 is a diagram illustrating one example of information stored in a reference information storage unit.

FIG. 6 is a diagram illustrating a first example of output information being output from an output unit to an evaluator terminal or a user terminal.

FIG. 7 is a diagram illustrating a second example of the output information being output from the output unit to the evaluator terminal or the user terminal.

FIG. 8 is a diagram illustrating a third example of the output information being output from the output unit to the evaluator terminal or the user terminal.

FIG. 9 is a diagram illustrating a fourth example of the output information being output from the output unit to the evaluator terminal or the user terminal.

FIG. 10 is a diagram illustrating a fifth example of the output information being output from the output unit to the evaluator terminal or the user terminal.

FIG. 11 is a diagram illustrating a hardware configuration example of the movement information generation apparatus.

FIG. 12 is a flowchart illustrating one example of processing executed by the movement information generation apparatus.

### DESCRIPTION OF EMBODIMENTS

An example embodiment of the present invention are described below with reference to the drawings. Note that, in all the drawings, a similar constituent element is denoted with a similar reference sign, and description therefor is omitted as appropriate.

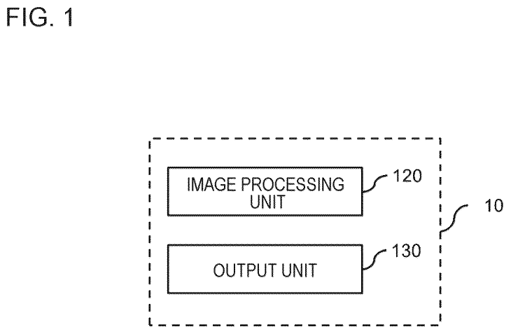

FIG. 1 is a diagram illustrating an overview of a movement information generation apparatus 10 according to an example embodiment. The movement information generation apparatus 10 is an apparatus that assists rehabilitation carried out by a subject, and includes an image processing unit 120 and an output unit 130. The image processing unit 120 generates first movement information indicating movement of a subject at first timing by processing a first image acquired by capturing the subject at the first timing. The output unit 130 determines a difference between the first movement information and comparison movement information indicating movement being a comparison target, and outputs output information being information relating to the difference.

According to the movement information generation apparatus 10, the output information relates to the difference between the first movement information indicating movement of a subject at the first timing and the comparison movement information indicating movement being a comparison target. Thus, a person who assists rehabilitation of a motor function of a subject or the subject can recognize effectiveness of rehabilitation for the subject at the first timing or the like by using the output information, and hence subsequent rehabilitation can be carried out appropriately.

A detailed example of the movement information generation apparatus 10 is described below.

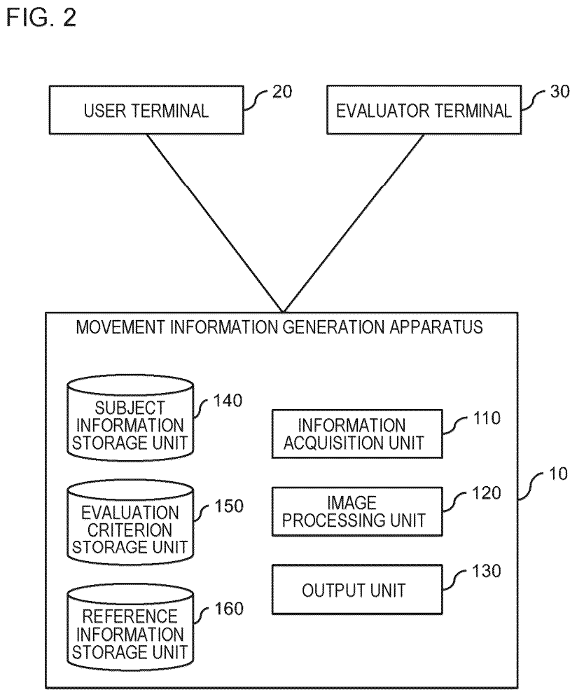

FIG. 2 is a diagram illustrating one example of a functional configuration of the movement information generation apparatus 10 along with a usage environment of the movement information generation apparatus 10. The movement information generation apparatus 10 is used together with a user terminal 20 and an evaluator terminal 30. At least a part of a movement information generation system is configured by the movement information generation apparatus 10, the user terminal 20, and the evaluator terminal 30. Note that, the movement information generation system may not include one of the user terminal 20 and the evaluator terminal 30.

The user terminal 20 is operated by a first therapist stationed at a facility for carrying out rehabilitation, or a subject. The evaluator terminal 30 is operated by a second therapist outside of the above-mentioned facility. The second therapist is more experienced as compared with the first therapist, and can provide advice to the first therapist. Note that, for example, each of the first therapist and the second therapist is a medical therapist such as a physical therapist and an occupational therapist.

The movement information generation apparatus 10 acquires various types of information from the user terminal 20. Hereinafter, the information being acquired from the user terminal 20 is referred to as input information. The input information includes a first image. The first image is generated after a subject carries out a menu being set as rehabilitation. Further, the movement information generation apparatus 10 generates output information by using the input information, and transmits the output information being generated to the evaluator terminal 30.

The evaluator terminal 30 displays or prints the output information. A therapist generates advice information for a subject with reference to the output information. The advice information includes at least a part of pieces of the output information. Further, the evaluator terminal 30 transmits the advice information to the movement information generation apparatus 10. The movement information generation apparatus 10 transmits the advice information to the user terminal 20, and also stores the advice information in a subject information storage unit 140.

Further, the user terminal 20 displays or prints the advice information. A subject or the first therapist decides a menu for subsequent rehabilitation with reference to the advice information. Further, the subject carries out the subsequent rehabilitation according to the menu.

Note that, the user terminal 20 may also function as the evaluator terminal 30. In this case, the advice information may not be transmitted or may be transmitted to the movement information generation apparatus 10.

Further, the movement information generation apparatus 10 includes an information acquisition unit 110, the image processing unit 120, the output unit 130, the subject information storage unit 140, an evaluation criterion storage unit 150, and a reference information storage unit 160. Note that, at least one of the subject information storage unit 140, the evaluation criterion storage unit 150, and the reference information storage unit 160 may be positioned outside of the movement information generation apparatus 10.

The information acquisition unit 110 acquires input information from the user terminal 20. Further, as required, the information acquisition unit 110 also acquires information from the evaluator terminal 30. As described above, the input information includes a first image. Note that, the input information may include other pieces of information. For example, the other pieces of information include at least one of the following.
A stage of rehabilitation. Hereinafter, referred to as stage information.
A comment from a subject. For example, a goal of rehabilitation (for example, a comment indicating what kind of movement is desired after rehabilitation
A comment from a family member of the subject
A comment from the first therapist. For example, a comment indicating a part in pain or a rehabilitation goal, or a question from the first therapist for the second therapist.

The image processing unit 120 generates first movement information by processing the first image included in the input information. As described with reference to FIG. 1, the first movement information indicates movement of the subject at the first timing at which the first image is generated. For example, when the first image includes a plurality of still images, for example, a plurality of frame images forming one moving image, the image processing unit 120 may generate skeleton information relating to the subject for each of the plurality of still images, and generate the first movement information by using a plurality of pieces of the skeleton information.

When the first image includes a plurality of frame images forming a moving image, the moving image may be generated while the subject undergoes a predetermined movement test. For example, the predetermined movement test is at least one of a timed up & go (TUG) test, a tandem gait test, and a one leg standing test.

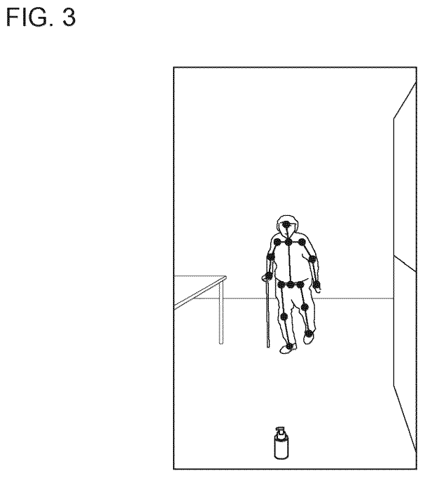

One example of the skeleton information is illustrated in FIG. 3. In this example, for example, the skeleton information indicates a relative position of different parts of a human body with respect to other parts thereof. For example, those parts are a head and a joint of a human, and a relative position is a line connecting those parts to each other. As one example, the part includes a head, a neck, a right shoulder, a left shoulder, a right elbow, a left elbow, a right wrist, a left wrist, a right waist, a left waist, a right knee, a left knee, a right ankle, and left ankle.

For example, the first movement information includes at least one of the following. In particular, when the image processing unit 120 generates the skeleton information, the image processing unit 120 can easily generate the following information.
A position of at least one joint point during specific movement
A motion vector of at least one joint point during specific movement
A relative position of at least two joint points during specific movement

For example, the “position of at least one joint point during specific movement” is a position of an ankle or a knee during walking. In this case, the first movement information indicates a degree at which a leg is lifted. The image processing unit 120 is also capable of generating the “position of at least one joint point during specific movement” by using one still image.

For example, the “motion vector of at least one joint point during specific movement” is a motion vector of at least one of an ankle, a wrist, a knee, and an elbow. In this case, the image processing unit 120 computes a position of a joint point in each of a plurality of frame images, and computes a motion vector by using a difference between the positions of the joint points in the plurality of frame images. Further, the image processing unit 120 is also capable of computing motion velocity of a joint point by using a frame rate.

Herein, the motion vector may be a motion vector of each of two adjacent joint points. As one example, the motion vector may be a motion vector of a right ankle and a motion vector of a right knee. The two motion vectors indicate motion of a right shin.

For example, the “relative position of at least two joint points during specific movement” is a relative position of a wrist and an ankle. In this case, the first movement information indicates body flexibility. The user terminal 20 is also capable of generating the “relative position of at least two joint points during specific movement” by using one still image.

As described with reference to FIG. 1, the output unit 130 determines a difference between the first movement information and comparison movement information, and outputs the output information being information relating to the difference.

For example, the comparison movement information is at least one of the following.

(1) Information generated by processing a second image acquired by capturing a subject at second timing prior to the first timing. In this case, for example, the comparison movement information is stored in the subject information storage unit 140.
(2) Information generated by processing a third image acquired by capturing a reference person different from the subject. In this case, for example, the comparison movement information is stored in the reference information storage unit 160.

In the item (1) given above, for example, the second timing is before starting a current rehabilitation menu. In this case, the difference between the first movement information and the comparison movement information indicates effectiveness of the current menu, in other words, a rehabilitation progress degree.

In the item (2) given above, for example, the reference person is a person without any physical impairments. In this case, for example, the comparison movement information indicates goal movement. Further, the difference between the first movement information and the comparison movement information indicates a difference in movement between the subject after rehabilitation and the reference person. In this case, the difference also indicates the rehabilitation progress degree.

The image processing unit 120 may generate the comparison movement information by processing the second image similarly to the first image. Moreover, the image processing unit 120 may generate the comparison movement information by processing the third image similarly to the first image.

Further, for example, the output information includes at least one of the following.

(a) Information indicating effectiveness of rehabilitation. For example, the rehabilitation progress degree. For example, the output unit 130 computes the progress degree by using a machine learning model using at least one of the first movement information, the comparison movement information, and the difference as an explanatory variable and the progress degree as an objective variable.
(b) Movement to be performed by the subject in a menu for subsequent rehabilitation. Hereinafter, the movement is referred to as recommended movement. For example, the output unit 130 decides the recommended movement, based on the difference described above. For example, the output unit 130 sets new movement, for example, movement with a high load as the recommended movement when rehabilitation progresses, and maintains current recommended movement when rehabilitation does not progress.
(c) The first movement information and the comparison movement information. One example of the first movement information is the skeleton information being generated by the image processing unit 120. In this case, the comparison movement information is also preferably the
(d) An input field for the second therapist
(e) At least one piece of information included in data. For example, at least one of a comment from the subject, a comment from a family member of the subject, and a comment from the first therapist.

In the item (b) given above, the first movement information is generated for each of a plurality of body parts in some cases. In this case, the comparison movement information is also prepared for each of a plurality of body parts. In this case, the output information includes the recommended movement for each of a plurality of body parts.

Further, rehabilitation includes a plurality of stages. Thus, the recommended movement is preferably set for each stage. In this case, in the item (a) given above, the output unit 130 preferably decides the recommended movement by using stage information included in the input information being acquired by the information acquisition unit 110.

Note that, the input information being acquired by the information acquisition unit 110 may include a plurality of pieces of first information, for example, a plurality of moving images. The plurality of the first images indicate the same movement performed by the same subject during the same period. In this case, the output unit 130 generates the first movement information from each of the plurality of the first images, and computes a difference between the comparison movement information and each of the plurality of pieces of the first movement information. Further, the output unit 130 may include, in the output information, a result acquired by subjecting the difference to statistical processing. One example of the statistical processing is computation of an average value, computation of a mode, or the like, but is not limited thereto. Herein, the output unit 130 may include, in the output information, a result acquired by subjecting at least a part of pieces of the first movement information to the statistical processing. Herein, for example, information subjected to the statistical processing is at least one of the position of at least one joint point during specific movement, the motion vector of at least one joint point during specific movement, and the relative position of at least two joint points during specific movement.

Further, the output unit 130 may display or print the output information in such a way that information based on past first movement information and information based on current first movement information can be compared with each other, for example, side by side. Herein, for example, the information to be compared with is at least one of the position of at least one joint point during specific movement, the motion vector of at least one joint point during specific movement, and the relative position of at least two joint points during specific movement. For example, by using the information based on the past first movement information and the information based on the current first movement information, the output unit 130 may include, in the output information, information visually indicating a change of an operation range or a pose of a skeleton, for example, a graph. For example, the pose is indicated by deviation from a proper position of a spine. In this manner, it becomes easier to recognize transition of a degree of physical recovery of the subject by carrying out rehabilitation. Note that, the information to be compared with may be a result acquired by subjecting those to the statistical processing.

For example, it is conceived a case where both the first movement information and the recommended movement are “front and back swing by a leg”. “Front and back swing by a leg” is movement where the subject is in a state of standing on one leg while holding onto a handrail or the like, and slowly moves the other leg back and forth to its maximum extent without using momentum. A purpose of this exercise is to promote smooth movement of a hip joint.

In this case, the image processing unit 120 computes a vertical operation range of a leg, based on the skeleton information. Further, the output unit 130 computes a difference between the vertical motion range of the leg being currently computed and a vertical operation range of the leg of the subject being computed in the past, and evaluates a motor function improvement amount by using the difference.

Note that, both the first movement information and the recommended movement may be “knee extension swing”. This movement is movement where the subject is in a state of seating in a chair, swings one leg up and down in small increments in a state of straightening the one leg. As velocity of movement increases, a load on the subject increases. Further, as the leg is raised higher, more muscles are moved. A purpose of this exercise is to stabilize movement of a knee.

In this case, the image processing unit 120 computes the number of times for which a leg is raised and lowered within a specified time, based on the skeleton information, and evaluates an exercise amount and a load amount.

The subject information storage unit 140 stores various pieces of information relating to the subject. Detailed examples of the information stored in the subject information storage unit 140 are described later with reference to the other drawings.

The evaluation criterion storage unit 150 stores information required for deciding the effectiveness of rehabilitation. For example, the evaluation criterion storage unit 150 stores a model for outputting the effectiveness of rehabilitation for each body part and each stage. For example, this model is generated by machine learning that uses at least one of the first movement information, the comparison movement information, and the difference as an explanatory variable and information indicating a rehabilitation progress degree as an objective variable. Herein, for example, the progress degree may be indicated by a progress rate (for example, %), or a binary value of completed/uncompleted.

The reference information storage unit 160 stores information required for generating the output information. One example of the information is information indicating the recommended movement. Detailed examples of the information stored in the reference information storage unit 160 are described later with reference to the other drawings.

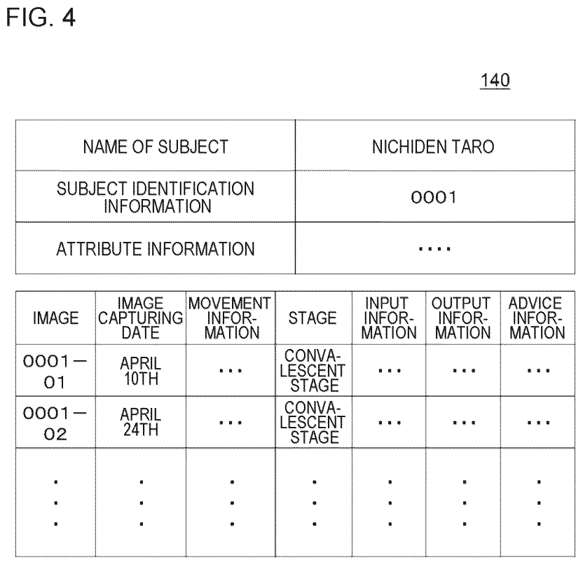

FIG. 4 is a diagram illustrating one example of information stored in the subject information storage unit 140. In the example illustrated in the present diagram, the subject information storage unit 140 stores at least one of the following.
A name of the subject
Subject identification information. One example of the information is a subject ID.
An attribute of the subject. For example, at least one of age, gender, a height, a weight, activities of daily living (Barthel index), grip strength, a one leg standing time, a result such as a required time for a TUG test, a result of a tandem gait test, a result of a one leg standing test, and a Berg balance scale of the subject. When the subject requires caring, the attribute further preferably includes a care level.
An image (first image) being acquired from the movement information generation apparatus 10 in the past, and movement information (first movement information) based thereon. Those are used as the second image and the comparison movement information.
A rehabilitation stage when the image is acquired
Information other than a stage among the pieces of the input information acquired along with the image
The output information relating to each image
The advice information relating to each image

Among the above-mentioned pieces of the information, the attribute of the subject may be updated regularly, for example, every three months.

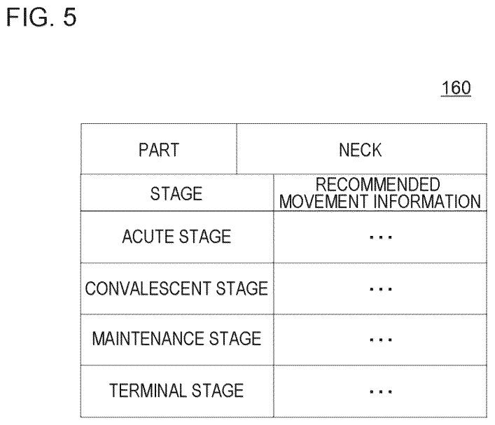

FIG. 5 is a diagram illustrating one example of information stored in the reference information storage unit 160. In the example illustrated in the present diagram, the reference information storage unit 160 stores information indicating the recommended movement for each body part. The information preferably includes text information describing the recommended movement and image information directly indicating the recommended movement. The image information includes at least one of a still image and a moving image. Further, in the example illustrated in the present diagram, the reference information storage unit 160 stores the information indicating the recommended movement for each body part and each rehabilitation stage.

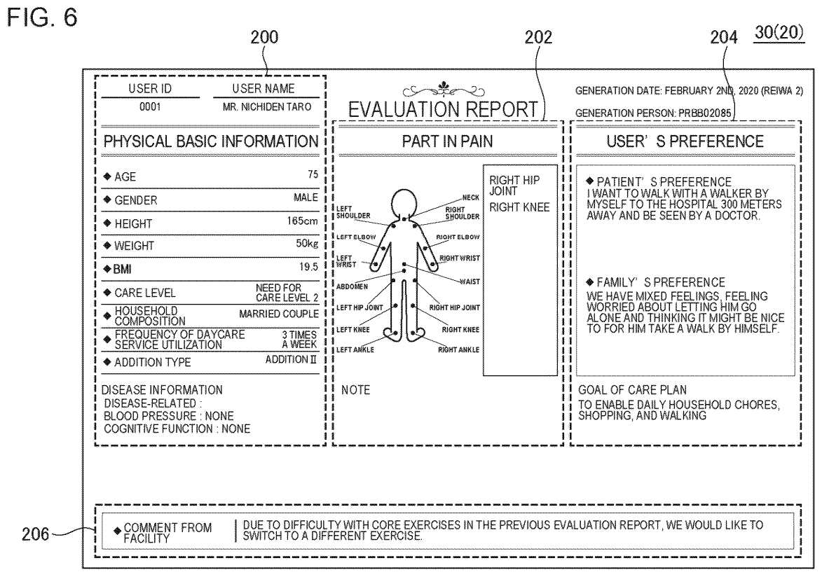

FIG. 6 is diagram illustrating a first example of the output information being output from the output unit 130 to the evaluator terminal 30 or the user terminal 20. In the example illustrated in the present diagram, the output information includes a first field 200 displaying basic information relating to a subject, a second field 202 indicating a problematic part of a body, for example, a part in pain, a third field 204 indicating a comment from the subject and a family member, and a fourth field 206 indicating a comment from the first therapist.

The output unit 130 acquires information to be indicated in the first field 200 from the subject information storage unit 140. Further, the output unit 130 generates information to be indicated in the second field 202, the third field 204, and the fourth field 206 by using the input information being acquired by the information acquisition unit 110.

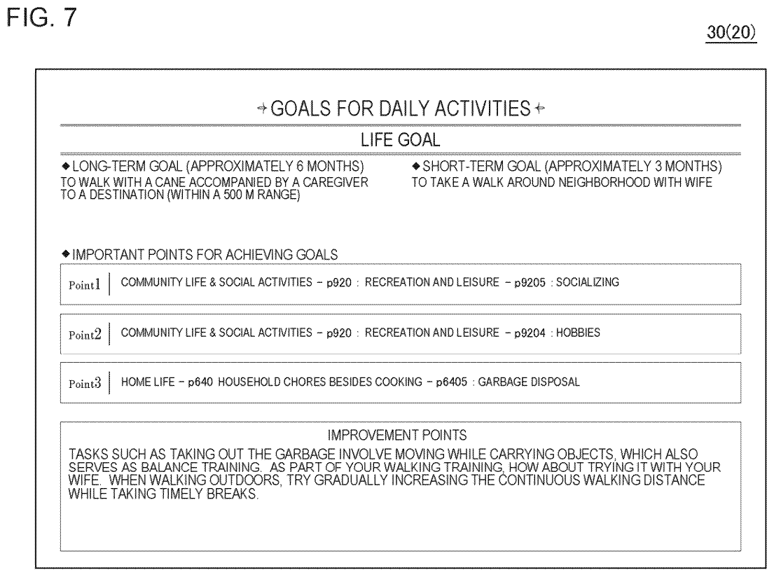

FIG. 7 is a diagram illustrating a second example of the output information being output from the output unit 130 to the evaluator terminal 30 or the user terminal 20. In the example illustrated in the present diagram, the output information includes a goal of rehabilitation and comprehensive advice for achieving the goal. For example, the output unit 130 generates information illustrated in the present diagram by using the input information being acquired by the information acquisition unit 110. Note that, the output unit 130 may acquire a part of pieces of the information illustrated in the present diagram from the subject information storage unit 140 or the reference information storage unit 160.

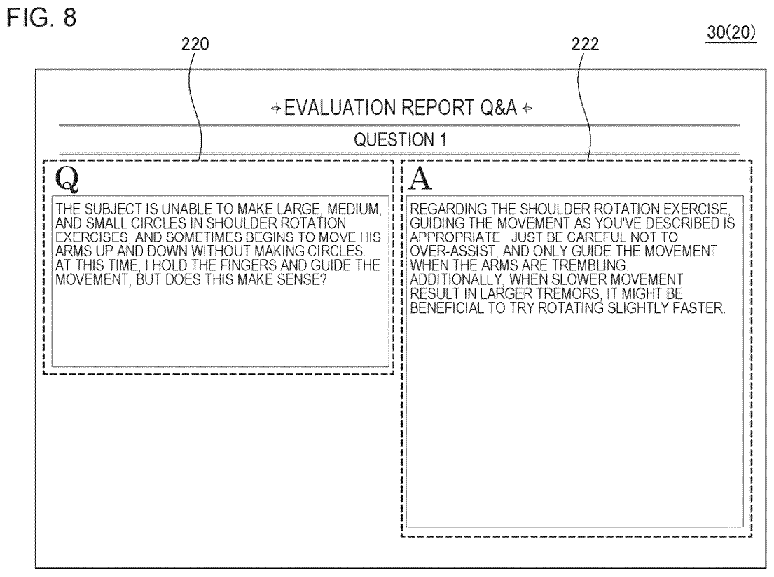

FIG. 8 is a diagram illustrating a third example of the output information being output from the output unit 130 to the evaluator terminal 30 or the user terminal 20. In the example illustrated in the present diagram, the output information includes a fifth field 220 displaying a question from the first therapist for the second therapist, and a sixth field 222 in which the second therapist inputs an answer therefor. Information being input in the sixth field 222 is transmitted, as a part of pieces of the advice information, from the evaluator terminal 30 or the user terminal 20 to the movement information generation apparatus 10.

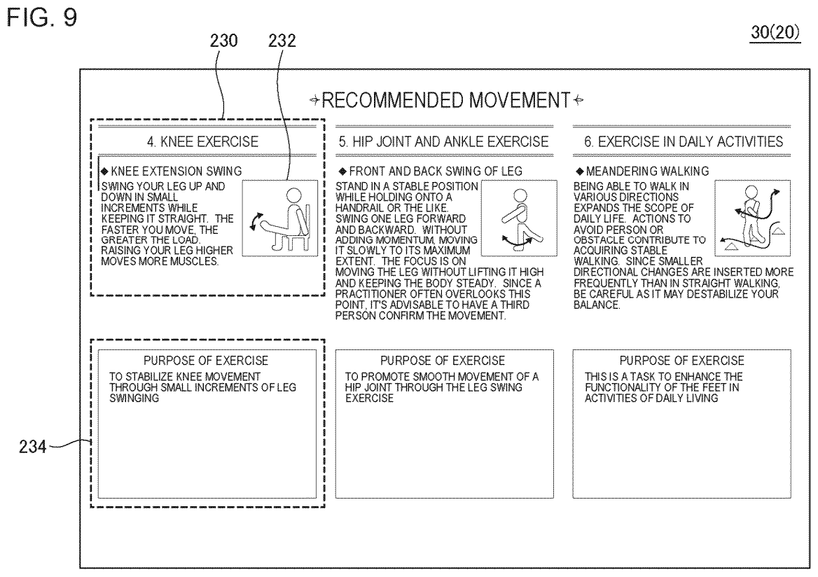

FIG. 9 is a diagram illustrating a fourth example of the output information being output from the output unit 130 to the evaluator terminal 30 or the user terminal 20. In the example illustrated in the present diagram, the output information includes a seventh field 230 indicating the recommended movement, and an eighth field 234 indicating a goal of the recommended movement. The output unit 130 acquires information indicated in the fields from the reference information storage unit 160. Herein, the eighth field 234 may be updatable by the second therapist. In this case, information after update is transmitted, as a part of pieces of the advice information, from the evaluator terminal 30 or the user terminal 20 to the movement information generation apparatus 10.

The seventh field 230 further includes a ninth field 232 displaying an image indicating the recommended movement. An image displayed in the ninth field 232 may be a still image or a moving image. In the former case, when the ninth field 232 is selected, the moving image may be reproduced in the ninth field 232.

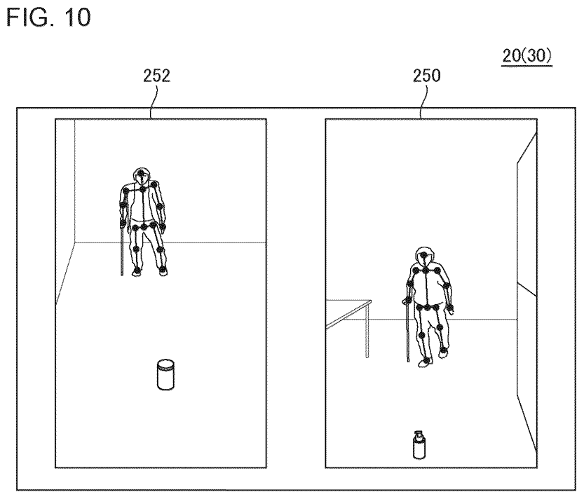

FIG. 10 is a diagram illustrating a fifth example of the output information being output from the output unit 130 to the evaluator terminal 30 or the user terminal 20. In the example illustrated in the present diagram, the output information includes a tenth field 250 indicating the first movement information, and an eleventh field 252 including the comparison movement information. In the tenth field 250, the skeleton information may be displayed to overlap with the first image. Further, in the eleventh field 252, the skeleton information may be displayed to overlap with an image from which the comparison movement information is generated, for example, the second image.

Note that, in each of the fields illustrated in FIG. 10, only the skeleton information may be displayed, or only the image may be displayed. In the former case, the skeleton information being the first movement information and the skeleton information being second movement information may be displayed in an overlapping manner.

Note that, at least a part of pieces of the output information illustrated in FIGS. 6 to 10 is corrected by the second therapist via the evaluator terminal 30, and then is transmitted, as at least a part of pieces of the advice information, to the movement information generation apparatus 10.

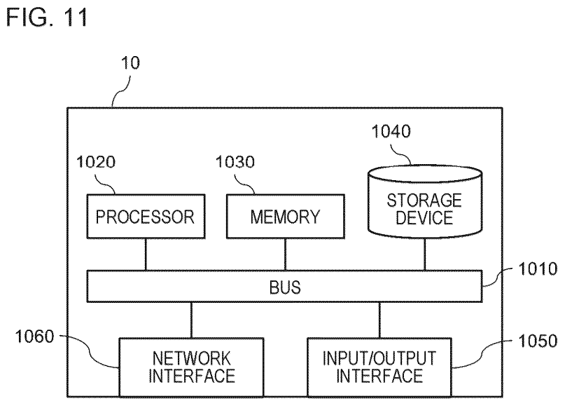

FIG. 11 is a diagram illustrating a hardware configuration of the movement information generation apparatus 10, the movement information generation apparatus 10 includes a bus 1010, a processor 1020, a memory 1030, a storage device 1040, an input/output interface 1050, and a network interface 1060.

The bus 1010 is a data transmission path in which the processor 1020, the memory 1030, the storage device 1040, the input/output interface 1050, and the network interface 1060 transmit and receive data mutually. However, a method of connecting the processor 1020 and the like to one another is not limited to bus connection.

The processor 1020 is a processor achieved by a central processing unit (CPU), a graphics processing unit (GPU), or the like.

The memory 1030 is a main storage apparatus achieved by a random access memory (RAM), or the like.

The storage device 1040 is an auxiliary storage apparatus achieved by a hard disk drive (HDD), a solid state drive (SSD), a removable medium such as a memory card, a read only memory (ROM), or the like. The storage device 1040 stores a program module achieving each of functions (for example, the information acquisition unit 110, the image processing unit 120, and the output unit 130) of the movement information generation apparatus 10. The processor 1020 reads each of the program modules on the memory 1030 and executes the read program module, and thereby each of the functions associated with each of the program modules is achieved. Further, the storage device 1040 also functions as at least one of the subject information storage unit 140, the evaluation criterion storage unit 150, and the reference information storage unit 160.

The input/output interface 1050 is an interface for connecting the movement information generation apparatus 10 and various input/output devices to each other.

The network interface 1060 is an interface for connecting the movement information generation apparatus 10 to a network. For example, the network is a local area network (LAN) or a wide area network (WAN). A method of connecting the network interface 1060 to the network may be wireless connection, or may be wired connection, the movement information generation apparatus 10 may communicate with the user terminal 20 and the evaluator terminal 30 via the network interface 1060.

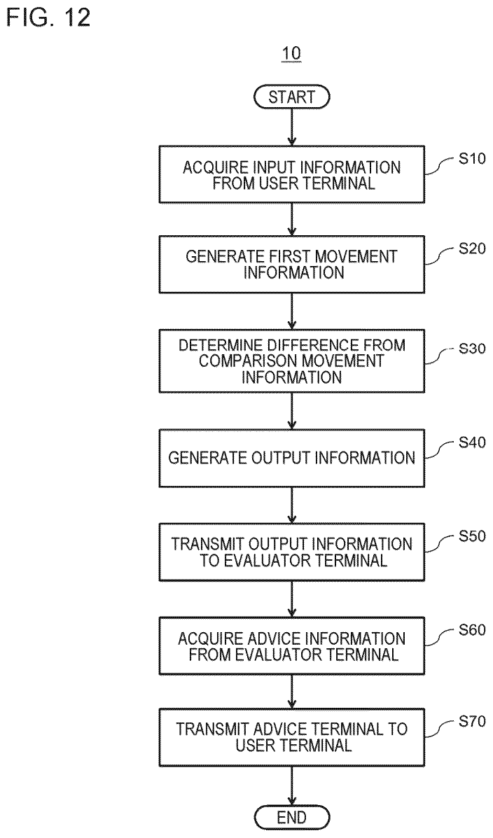

FIG. 12 is a flowchart illustrating one example of processing executed by the movement information generation apparatus 10. First, a user of the user terminal 20 inputs required information. Further, the user terminal 20 transmits the information as input information to the movement information generation apparatus 10. As described above, the input information includes a first image. Herein, the input information may include a plurality of the first images. In this case, the plurality of the first images, for example, a plurality of moving images may be included. The plurality of the first images indicate the same movement performed by the same subject.

The information acquisition unit 110 of the movement information generation apparatus 10 acquires the input information (step S10). Further, the image processing unit 120 generates first movement information by processing the first image (step S20). Herein, the image processing unit 120 may generate the first movement information from each of the plurality of the first images.

Subsequently, the output unit 130 reads out comparison movement information from the subject information storage unit 140 or the reference information storage unit 160, and determines a difference between the comparison movement information being read out and the first movement information (step S30). Further, the output unit 130 generates output information relating to the difference. Specific examples of the output information are as described with reference to FIG. 2, and FIGS. 6 to 10. In this state, the output unit 130 may generate information indicating a rehabilitation progress degree by using information stored in the evaluation criterion storage unit 150, and include the information in the output information (step S40). Herein, when the first movement information is generated from each of the plurality of the first images, the output unit 130 may include a result of statistical processing in the output information.

Further, the output unit 130 outputs the output information to the evaluator terminal 30, and stores the output information in the subject information storage unit 140. Herein, the output unit 130 may output the output information to the user terminal 20 (step S50).

The second therapist confirms the output information by using the evaluator terminal 30. Further, the second therapist generates advice information by using the output information. For example, the second therapist generates the advice information by processing at least a part of pieces of the output information. Further, the evaluator terminal 30 transmits the advice information to the movement information generation apparatus 10.

The information acquisition unit 110 of the movement information generation apparatus 10 acquires the advice information (step S60). Further, the information acquisition unit 110 transmits the advice information to the user terminal 20, and stores the advice information in the subject information storage unit 140 (step S70).

As described above, according to the present example embodiment, output information relates to a difference between first movement information indicating movement of a subject at first timing and comparison movement information indicating movement being a comparison target. Thus, a person who assists rehabilitation of the subject or the subject can carry out rehabilitation as appropriate by using the output information. As one example, the second therapist can easily generate advice information by using the output information. Further, a person who assists rehabilitation of a subject or the subject can carry out rehabilitation, based on advice from the second therapist, in other words, an experienced therapist.

Further, the second therapist is not required to a facility where the subject is present. Thus, the second therapist can handle a larger number of subjects.

While the example embodiment of the present invention is described above with reference to the drawings, these are examples of the present invention, and various configurations other than those described above may be adopted.

Further, in the plurality of flowcharts used in the description given above, a plurality of steps (pieces of processing) are described in order, but the execution order of the steps executed in each of the example embodiments is not limited to the described order. In each of the example embodiments, the order of the illustrated steps can be changed without interfering with the contents. Further, the example embodiments described above may be combined with each other within a range where the contents do not conflict with each other.

The whole or a part of the example embodiment described above can be described as, but not limited to, the following supplementary notes.

1. A movement information generation apparatus, including:
an image processing unit that generates first movement information indicating movement of a subject at first timing by processing a first image acquired by capturing the subject at the first timing; and
an output unit that determines a difference between the first movement information and comparison movement information indicating movement being a comparison target, and outputs output information being information relating to the difference.
   2. The movement information generation apparatus according to supplementary note 1, wherein the comparison movement information is generated by processing a second image acquired by capturing a subject at second timing prior to the first timing.
   3. The movement information generation apparatus according to supplementary note 1, wherein the comparison movement information is generated by processing a third image acquired by capturing a reference person being a person different from the subject.
   4. The movement information generation apparatus according to supplementary note 2, wherein the image processing unit generates the comparison movement information by processing the second image.
   5. The movement information generation apparatus according to any one of supplementary notes 1 to 4, wherein
the first timing is timing after the subject performs rehabilitation training, and
the output information includes recommended movement being movement to be performed by the subject.
   6. The movement information generation apparatus according to supplementary note 5, wherein
the difference is generated for each of a plurality of parts of a body, and
the output information includes the recommended movement for each of the plurality of parts.
   7. The movement information generation apparatus according to supplementary note 5 or 6, further including
an information acquisition unit that acquires stage information indicating a stage of rehabilitation training of the subject, wherein
the output unit decides the recommended movement by using the stage information.
   8. The movement information generation apparatus according to any one of supplementary notes 5 to 7, wherein
the output information includes the first movement information and the comparison movement information.
   9. The movement information generation apparatus according to any one of supplementary notes 1 to 8, wherein
the first image includes a plurality of still images, and
the image processing unit generates skeleton information relating to the subject for each of the plurality of still images, and generates the first movement information by using the skeleton information.
   10. The movement information generation apparatus according to any one of supplementary notes 1 to 9, wherein
the first movement information includes at least one of the following
   a position of at least one joint point during specific movement,
   a motion vector of at least one joint point during specific movement, and
   a relative position of at least two joint points during specific movement.
      11. A movement information generation system including:
a terminal; and
a movement information generation apparatus, wherein
the movement information generation apparatus includes
   an image processing unit that generates first movement information indicating movement of a subject at first timing by processing a first image acquired by capturing the subject at the first timing, and
   an output unit that determines a difference between the first movement information and comparison movement information indicating movement being a comparison target, and outputs, to the terminal, output information being information relating to the difference, and
the terminal displays or prints the output information.
   12. The movement information generation system according to supplementary note 11, wherein
the comparison movement information is generated by processing a second image acquired by capturing a subject at second timing prior to the first timing.
   13. The movement information generation system according to supplementary note 11, wherein
the comparison movement information is generated by processing a third image acquired by capturing a reference person being a person different from the subject.
   14. The movement information generation system according to supplementary note 12, wherein
the image processing unit generates the comparison movement information by processing the second image.
   15. The movement information generation system according to any one of supplementary notes 11 to 14, wherein
the first timing is timing after the subject performs rehabilitation training, and
the output information includes recommended movement being movement to be performed by the subject.
   16. The movement information generation system according to supplementary note 15, wherein
the difference is generated for each of a plurality of parts of a body, and
the output information includes the recommended movement for each of the plurality of parts.
   17. The movement information generation system according to supplementary note 15 or 16, further including
an information acquisition unit that acquires stage information indicating a stage of rehabilitation training of the subject, wherein
the output unit decides the recommended movement by using the stage information.
   18. The movement information generation system according to any one of supplementary notes 15 to 17, wherein
the output information includes the first movement information and the comparison movement information.
   19. The movement information generation system according to any one of supplementary notes 11 to 18, wherein
the first image includes a plurality of still images, and
the image processing unit generates skeleton information relating to the subject for each of the plurality of still images, and generates the first movement information by using the
   20. The movement information generation system according to any one of supplementary notes 11 to 19, wherein
the first movement information includes at least one of the following
   a position of at least one joint point during specific movement,
   a motion vector of at least one joint point during specific movement, and
   a relative position of at least two joint points during specific movement.
      21. A movement information generation method including, by a computer:
generating first movement information indicating movement of a subject at first timing by processing a first image acquired by capturing the subject at the first timing; and
determining a difference between the first movement information and comparison movement information indicating movement being a comparison target, and outputting output information being information relating to the difference.
   22. The movement information generation method according to supplementary note 21, wherein
the comparison movement information is generated by processing a second image acquired by capturing a subject at second timing prior to the first timing.
   23. The movement information generation method according to supplementary note 21, wherein
the comparison movement information is generated by processing a third image acquired by capturing a reference person being a person different from the subject.
   24. The movement information generation method according to supplementary note 22, further including,
by the computer,
   generating the comparison movement information by processing the second image.
      25. The movement information generation method according to any one of supplementary notes 21 to 24, wherein
the first timing is timing after the subject performs rehabilitation training, and
the output information includes recommended movement being movement to be performed by the subject.
   26. The movement information generation method according to supplementary note 25, wherein
the difference is generated for each of a plurality of parts of a body, and
the output information includes the recommended movement for each of the plurality of parts.
   27. The movement information generation method according to supplementary note 25 or 26, further including,
by the computer:
   acquiring stage information indicating a stage of rehabilitation training of the subject; and
   deciding the recommended movement by using the stage information.
      28. The movement information generation method according to any one of supplementary notes 25 to 27, wherein
the output information includes the first movement information and the comparison movement information.
   29. The movement information generation method according to any one of supplementary notes 21 to 28, wherein
the first image includes a plurality of still images,
the movement information generation method further including,
by the computer,
   generating skeleton information relating to the subject for each of the plurality of still images, and generating the first movement information by using the skeleton information.
      30. The movement information generation method according to any one of supplementary notes 21 to 29, wherein
the first movement information includes at least one of the following
   a position of at least one joint point during specific movement,
   a motion vector of at least one joint point during specific movement, and
   a relative position of at least two joint points during specific movement.
      31. A computer-readable storage storing a program causing a computer to execute:
image processing of generating first movement information indicating movement of a subject at first timing by processing a first image acquired by capturing the subject at the first timing; and
output processing of determining a difference between the first movement information and comparison movement information indicating movement being a comparison target, and outputting output information being information relating to the difference.
   32. The storage medium according to supplementary note 31, wherein
the comparison movement information is generated by processing a second image acquired by capturing a subject at second timing prior to the first timing.
   33. The storage medium according to supplementary note 31, wherein
the comparison movement information is generated by processing a third image acquired by capturing a reference person being a person different from the subject.
   34. The storage medium according to supplementary note 32, wherein
the image processing function generates the comparison movement information by processing the second image.
   35. The storage medium according to any one of supplementary notes 31 to 34, wherein
the first timing is timing after the subject performs rehabilitation training, and
the output information includes recommended movement being movement to be performed by the subject.
   36. The storage medium according to supplementary note 35, wherein
the difference is generated for each of a plurality of parts of a body, and
the output information includes the recommended movement for each of the plurality of parts.
   37. The storage medium according to supplementary note 35 or 36, storing the program further causing the computer to execute
information acquisition function of acquiring stage information indicating a stage of rehabilitation training of the subject, wherein
the output function decides the recommended movement by using the stage information.
   38. The storage medium according to any one of supplementary notes 35 to 37, wherein
the output information includes the first movement information and the comparison movement information.
   39. The storage medium according to any one of supplementary notes 31 to 38, wherein
the first image includes a plurality of still images, and
the image processing function generates skeleton information relating to the subject for each of the plurality of still images, and generates the first movement information by using the skeleton information.
   40. The storage medium according to any one of supplementary notes 31 to 39, wherein
the first movement information includes at least one of the following
   a position of at least one joint point during specific movement,
   a motion vector of at least one joint point during specific movement, and
   a relative position of at least two joint points during specific movement.
      40. The program according to any one of supplementary notes 31 to 40.

### REFERENCE SIGNS LIST

10 Movement information generation apparatus
20 User terminal
30 Evaluator terminal
110 Information acquisition unit
120 Image processing unit
130 Output unit
140 Subject information storage unit
150 Evaluation criterion storage unit
160 Reference information storage unit

What is claimed is:

1. A movement information generation apparatus comprising:
at least one memory configured to store instructions; and
at least one processor configured to execute the instructions to preform operations comprising:
generating first movement information indicating movement of a subject at first timing by processing a first image acquired by capturing the subject at the first timing; and
determining a difference between the first movement information and comparison movement information indicating movement being a comparison target, and outputting output information being information relating to the difference.

2. The movement information generation apparatus according to claim 1, wherein
the comparison movement information is generated by processing a second image acquired by capturing a subject at second timing prior to the first timing.

3. The movement information generation apparatus according to claim 1, wherein
the comparison movement information is generated by processing a third image acquired by capturing a reference person being a person different from the subject.

4. The movement information generation apparatus according to claim 2, wherein
generating the comparison movement information by processing the second image.

5. The movement information generation apparatus according to claim 1, wherein
the first timing is timing after the subject performs rehabilitation training, and
the output information includes recommended movement being movement to be performed by the subject.

6. The movement information generation apparatus according to claim 5, wherein
the difference is generated for each of a plurality of parts of a body, and
the output information includes the recommended movement for each of the plurality of parts.

7. The movement information generation apparatus according to claim 5, wherein
acquiring stage information indicating a stage of rehabilitation training of the subject,
deciding the recommended movement by using the stage information.

8. The movement information generation apparatus according to claim 1, wherein
the output information includes the first movement information and the comparison movement information.

9. The movement information generation apparatus according to claim 1, wherein
the first image includes a plurality of still images, and
generating skeleton information relating to the subject for each of the plurality of still images, and generates the first movement information by using the skeleton information.

10. The movement information generation apparatus according to claim 1, wherein
the first movement information includes at least one of the following
a position of at least one joint point during specific movement,
a motion vector of at least one joint point during specific movement, and
a relative position of at least two joint points during specific movement.

11. (canceled)

12. A movement information generation method comprising,
by a computer:
generating first movement information indicating movement of a subject at first timing by processing a first image acquired by capturing the subject at the first timing; and
determining a difference between the first movement information and comparison movement information indicating movement being a comparison target, and outputting output information being information relating to the difference.

13. A non-transitory computer-readable storage medium storing a program for causing a computer to perform operations comprising:
generating first movement information indicating movement of a subject at first timing by processing a first image acquired by capturing the subject at the first timing; and
determining a difference between the first movement information and comparison movement information indicating movement being a comparison target, and outputting output information being information relating to the difference.

14. The movement information generation apparatus according to claim 5, wherein
the recommended movement is optimized to the subject.

15. The movement information generation apparatus according to claim 1, wherein
outputting a rehabilitation progress degree computed by machine learning model.

16. The movement information generation apparatus according to claim 1, wherein
the subject is injured or diseased individual.
